(19) United States
(12) Patent Application Publication
(10) Pub. No.: US 2008/0051621 A1
(43) Pub. Date: Feb. 28, 2008

(54) Magnetic Device

(75) Inventors: John Vines, Queensland (AU); George Ross, Queensland (AU)

Correspondence Address: SHOEMAKER AND MATTARE, LTD, 10 POST OFFICE ROAD - SUITE 110, SILVER SPRING, MD, 20910, US

(73) Assignee: GT & MJ HOLDINGS PTY LTD, Pacific Pines, Gold Coast, QLD (AU)

(21) Appl. No.: 11/575,085

(22) PCT Filed: Sep. 9, 2005
(86) PCT No.: PCT/AU05/01371

§ 371 Date: Mar. 12, 2007

(30) Foreign Application Priority Data
Sep. 10, 2004 (AU) 2004905197
Nov. 3, 2004 (AU) 2004100943

(51) Int. Cl. A61N 2/08

(52) U.S. Cl. 600/15

(57) ABSTRACT

The present invention is directed to a magnetic device for treating conditions such as incontinence, upper and lower back pain, irritable bowel syndrome, arthritis in the hand and fingers, menstruation problems and prostate problems. The magnetic device in a preferred form consists of a mesh with spaced apart holding portions for magnets so that the device produces a synergistic magnetic effect to the desired area. The magnetic device in another preferred form consists of spaced apart magnets that are arranged about a desired area for treatment but maintaining the spacing relative to each other. In another preferred form, the magnetic device is a garment overlay which has spaced apart magnets on the overlay in positions that correspond to significant acupuncture points when the overlay is fitted. The garment overlay also has locating means for locating the position of the overlay relative to the desired body portion.

### FIELD OF INVENTION

The present invention relates to methods and devices associated with the use of magnets and magnetism. The present invention has particular but not exclusive application for treatment of medical conditions. Reference to medical treatment in the body of the specification is by way of example only and the invention may have application in a variety of fields.

### BACKGROUND OF THE INVENTION

Magnets and magnetic therapy has been used to treat pain, incontinence and other urinary medical conditions. Magnets have been incorporated into clothing to subject a person to magnetic fields for a prolonged period time in order to improve their medical condition. Several patent specifications including U.S. Pat. No. 6,332,862, U.S. Pat. No. 5,950,239, CA02309479, CN1190554, CN1146365, CN1141146, and JP2002054002 disclose articles of clothing where magnets or magnetic elements have been sown or incorporated into the clothing. These patent specifications have been mainly focused on subjecting the body or body portion(s) with as many magnets or magnetic elements to apply magnetic therapy without due consideration for the arrangement and orientation of the magnets or magnetic elements.

Furthermore, incorporating magnets into clothing has limitations in that the clothing must be regularly washed and cleaned thereby subjecting the magnets or magnetic elements to damage or loss of magnetism. As well, a person is not being treated while their clothing with the magnets is being washed and cleaned unless they have magnets incorporated in all their garments. With washing, the magnets may be flipped and present an alternative orientation. In addition, as the clothing becomes worn, the spatial arrangement of the magnets or magnetic elements relative to each other changes. As a consequence of these changes including orientation change, the clothing incorporating the magnets or magnetic elements may not be providing the desired therapeutic benefits and may even be detrimental to the person wearing the clothing.

### OBJECT OF THE INVENTION

It is an object of the present invention to provide a device that overcomes at least in part one or more of the abovementioned disadvantages.

### SUMMARY OF THE INVENTION

In one aspect the present invention broadly resides in a device for use in applying a magnetic treatment, including:

a plurality of magnets; and

a holding member for holding and maintaining said plurality of magnets in a preset spaced apart arrangement relative to each other, wherein the location and orientation of the magnets relative to each other produces a synergistic effect.

Preferably the holding member is made of rigid but resiliently deformable plastic which is able to maintain the orientation and relative spacing of the magnets but is able to bend or be shaped for the application.

The plastic is preferably of inert material such as polypropylene or other suitable polyolefin. Preferably the holding member is made of suitable plastic that can be irradiated for sterilization and contain fire retardant polymers.

In one preferred embodiment the holding member is an elongate planar plastic member with magnet holding portions spaced apart at opposing ends. The elongate plastic member is preferably a modular unit where two or more members can be magnetically attached to form a beneficial arrangement.

Each holding portion may hold one or more magnets depending on the dose required for treatment or application. The spacing between the holding portions is preferably dependent on the magnetic field produced by the retained magnet(s). Preferably, the greater the magnetic field of the retained magnets, greater is the spacing between the holding portions. Preferably, the magnitude of the created magnetic fields is proportional to the spacing between the holding portions.

The magnets may be sealed within the holding portion. Alternatively, the magnets may be locatable in a holding portion that forms a closable pocket or a two-part holder. Where the holding portion is a closable pocket or a two-part holder, magnets may be changed or two or more magnets may be located in a single holding portion to alter the dosage.

In another preferred embodiment the device may include two or more elongate planar modules formed in a desired arrangement for application to a particular situation or condition. The elongate planar modules are arranged by using the attraction of opposing magnetic poles of each magnet so that one magnet in a holding member abuts another magnet in a second holding member. By way of example, a concertina arrangement of holding members can be formed and used to assist in the healing of a wound.

In a further preferred embodiment the holding member is a mesh with two or more holding portions spaced apart from each other to achieve the desired synergistic effect. The mesh preferably is shaped to suit the particular applications. The mesh may also have fastening means such as ties, studs or hook and eye fasteners such as Velcro to maintain a particular shape in the desired application. The mesh is preferably made of the same material as the elongate planar module. The mesh is preferably breathable and has a plurality of apertures to allow the passage of air.

The magnets are preferably formed from material that has a high magnetic flux density such as rare earth magnets. More preferably, the magnets are formed from Neodymium and Ferrite material or composites thereof. In an alternative other ferromagnetic material may be used including Aluminum-Nickel-Cobalt and Samarium-Cobalt. Alternatively the magnets may be made of ceramics.

The magnets are preferably disc shaped with one magnetic pole on one side of the disc and the other magnetic pole on the opposing side of the disc. Each disc is preferably substantially 25 mm in diameter and 2.2 mm thick.

Preferably the magnets have different shapes and strengths to suit particular applications and different body portion shapes.

The device preferably has one or more pockets for the placement of holding members and or magnets in position to treat particular areas.

The device in the form of a magnetic mesh can preferably serve as an overlay that may be positioned on or about a body portion in order to apply treatment.

In a preferred embodiment the device has an elastic portion to retain the position of the device relative to a particular body portion being treated. Preferably the elastic portion is a wide band.

In another aspect, the invention broadly resides in a method of using the abovementioned device for treatment including:

positioning the device on or about a body or body portion in a manner that operatively aligns the magnetic field with one or more acupuncture points; and

subjecting the body or body portion to the magnetic field of the magnets within the device for a set period of time.

The acupuncture points are preferably points in a body or body portion that are sensitive to magnetic fields. Acupuncture points may include areas where there are meridians of electrical energy extending along the body or body portion.

In another aspect the invention broadly resides in a system for treating with magnets including

a plurality of magnets;

holding means for holding the magnets in a preset spaced apart arrangement relative to each other, wherein the location and orientation of the magnets relative to each other produces a synergistic effect;

application means for positioning the holding means in an arrangement that allows the magnets to provide the synergistic effect and treat the desired area.

In a further aspect the present invention broadly resides in a magnetic device for applying a magnetic treatment, including

an adjustable member for adjusting the device about a body portion;

locating means for use in positioning the device about the body portion to achieve therapeutic benefit;

two or more magnets fixed to an overlay portion, each of said magnets are supported by a stiffener member; said overlay portion is adapted to wear over undergarments, wherein said overlay portion and adjustable member cooperate to wear the device over undergarments.

In another aspect the present invention broadly resides in a garment overlay for applying a magnetic treatment including

an adjustable member for adjusting the overlay about a body portion;

locating means for use in positioning the overlay about the body portion to achieve therapeutic benefit;

two or more magnets fixed to an overlay portion, each of said magnets are supported by a stiffener member; said overlay portion is adapted to wear over undergarments, wherein said overlay portion and adjustable member cooperate to wear the overlay over undergarments.

In another aspect the present invention broadly resides in a garment overlay for applying a magnetic treatment including

an adjustable member for adjusting the overlay about a body portion;

locating means for use in positioning the overlay about the body portion to achieve therapeutic benefit;

a holding member for holding and maintaining a plurality of magnets in a preset spaced apart arrangement relative to each other, wherein the location and orientation of the magnets relative to each other produces a synergistic effect, said holding member is fixed to an overlay portion, said holding member is substantially stiff; said overlay portion is adapted to wear over undergarments, wherein said overlay portion and adjustable member cooperate to wear the overlay over undergarments.

Said overlay portion preferably serves only to hold the magnets in position.

Said overlay portion preferably comprises of minimal material.

Said adjustable member preferably is a band that can be lengthened or shortened depending on the size of the body portion. Preferably the band is lengthened or shortened by means of adjustable fasteners. Where the body portion is the waist of the person, the band is preferably lengthened or shortened by means of detachable clips. Preferably the band has a plurality of clips to provide the wearer with a choice of adjustment. Preferably the plurality of clips form a series in alignment on the band. More preferably the clips are press studs.

The adjustable band may also be elasticized to assist with maintaining the desired position.

Alternately the band may have hook and eye fasteners such as Velcro for adjusting the band.

In a preferred embodiment the band can be opened to provide two free ends. In another preferred embodiment the band can include one or more portions that can be separated from each other. Where the band include one or more portions, insert portions may be added to lengthen the band.

The locating means may be associated with the band. Where the garment overlay is for overlaying underpants, the locating means is preferably a band shaped for locating over the wearer's hip bone. The adjustable member is preferably a band that can be lengthened or shortened by means of adjustable fasteners, and the locating means is the band shaped for locating the overlay over the wearer's hip bone.

The garment overlay may be made of suitable plastics, cloth or other suitable fabric.

In another aspect, the invention broadly resides in a garment overlay for positioning over underpants or similar undergarment worn by a wearer, including

an adjustable band that has a plurality of fasteners for lengthening or shortening the band; and

an overlay portion shaped to cover the lower torso region without covering the legs, a plurality of magnets fixed to the overlay portion and supported in position by a stiffener, wherein said band is shaped to fit around a wearer's waist and over the wearer's hip bones for location purposes to align and orientate said magnets.

Preferably there is at least one pair of magnets laterally spaced apart and the spacing is maintained by the stiffener. Preferably there are three pairs of laterally spaced apart magnets on a front section of the overlay portion. Preferably there are four pairs of laterally spaced apart magnets on a rear section of the overlay portion. Preferably there is a single or a pair of magnets fixed at the gusset section of the overlay portion. More preferably there are three pairs of laterally spaced apart magnets on a front section of the overlay portion, four pairs of laterally spaced apart magnets on a rear section of the overlay portion and there is a single or pair of magnets fixed at the gusset section of the overlay portion.

The overlay portion is preferably in the shape of a high leg-cut type garment with ample material to overlay an underpants.

### BRIEF DESCRIPTION OF THE DRAWINGS

In order that the present invention can be more readily understood and put into practical effect, reference will now be made to the accompanying drawings wherein:

FIGS. 1A-1D are diagrammatic views of a sheet of magnetic mesh of the first preferred embodiment of the invention;

FIG. 2 is a diagrammatic view of the elongate planar module of the second preferred embodiment of the invention;

FIG. 3 is a diagrammatic view of a triangular arrangement of three elongate planar modules as shown in FIG. 2;

FIG. 4 is a diagrammatic view of a concertina pattern arrangement of elongate planar modules of the second preferred embodiment;

FIG. 5 is a diagrammatic view of a device of magnetic mesh for use in treating backs;

FIGS. 6A-6B are diagrammatic views of a device of magnetic mesh for treatment of incontinence;

FIG. 7 is a diagrammatic view of the use of the device having a concertina pattern arrangement shown in FIG. 4 in treating a wound;

FIG. 8 is a diagrammatic view of a stiffener for a magnetic garment overlay;

FIG. 9 is a diagrammatic view of the front of the magnetic garment overlay;

FIG. 10 is a diagrammatic view of magnetic glove;

FIG. 11 is a diagrammatic view of the front of an alternative incontinence garment overlay;

FIG. 12 is a diagrammatic view of the rear of the alternative incontinence garment overlay;

FIG. 13 is an diagrammatic view of an alternative garment overlay; and

FIG. 14 is a diagrammatic rear view of a garment overlay for treatment of upper and lower back pain.

### DETAILED DESCRIPTION OF THE PREFERRED EMBODIMENTS

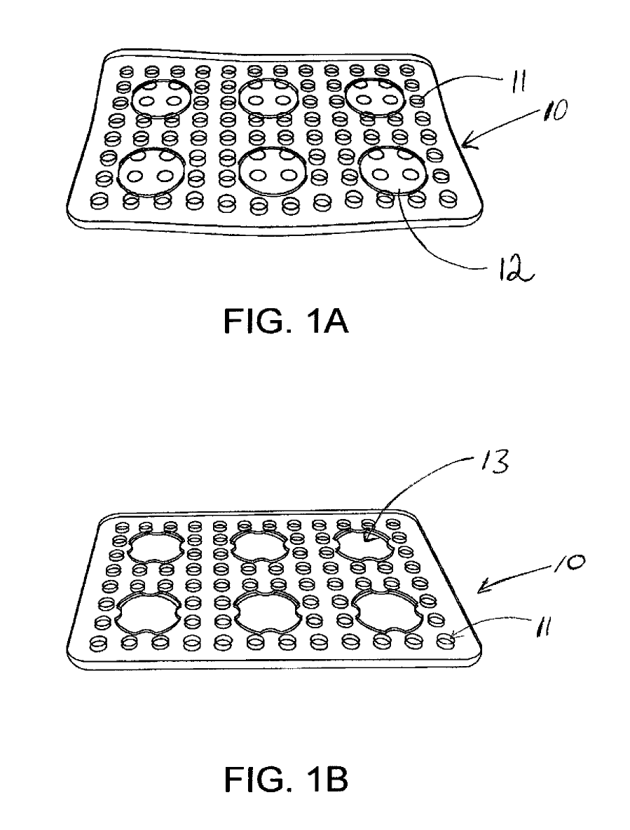

With reference to FIGS. 1A, 1B, 1C and 1D, there is shown a sheet of magnetic mesh 10 with apertures 11 allowing the circulation of air. The magnet holders 12 of FIG. 1A are sealed holders enclosing one or more disc shaped magnets (not shown). FIGS. 1B-1D show two-part magnet holders 13 having a scalloped top portion 14 snap-fitting into a base portion 15. The top portions 15 can be removed to replace or add magnets. The magnet holders 12,13 are fixed in position and spatial arranged to provide a synergistic magnetic field effect. The magnets are disc shaped having a 25 mm diameter and 2.2 mm thickness. However as people react differently and have a variety of shapes and sizes, the magnet sizes may differ between 8 to 30 mm diameter and 1.3 to 7 mm thickness. The magnets are composed of Neodymium Ferrite.

Each magnet has a magnetic strength of between 25 and 20000 gauss. The magnetic strength of the magnets used with a person will depend on the age and weight of the person, the area being treated and the therapeutic dose for the treatment. The magnetic strength may be increased if the treatment is not working or a larger area requires treatment by replacing the magnet(s) with more powerful magnets or adding additional magnets thereby increasing the magnetic field strength.

Each magnet has a north pole on one side of the disc and a south pole on the other side of the magnet. The magnets may be color coded with the north pole side colored one color while the side with the south pole colored with another color. By way of example the side with the north pole is colored blue and the south pole side is colored red. The magnets may be coated to protect them from corrosive materials.

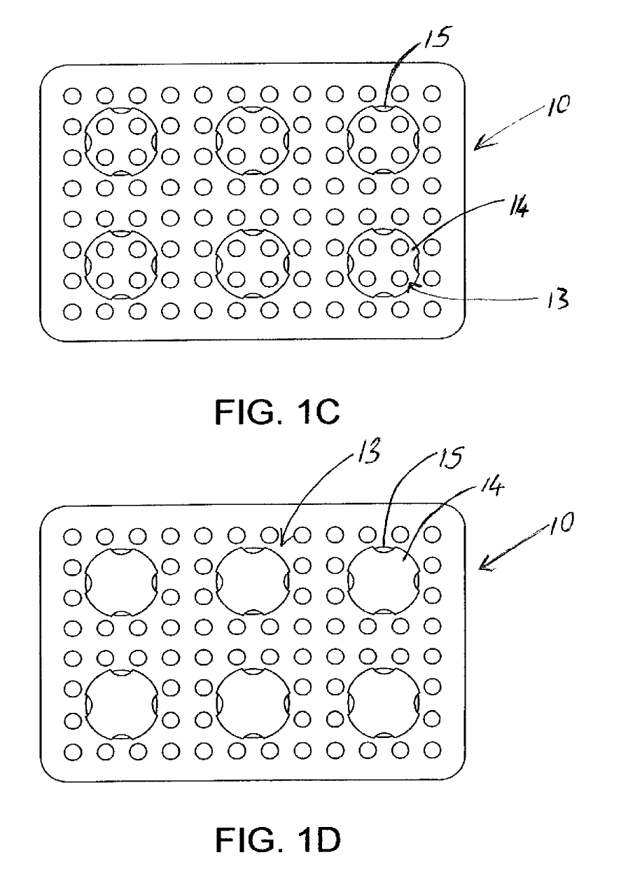

With reference to FIG. 2, there is shown an elongate planar modular member 20 with magnet holders 21 on opposing ends. The modular member 20 has the same type of magnet holders 21 as those of the mesh 10. The same type of magnets are also employed. Both the mesh 10 and modular member 20 are preferably made of plastics such as polypropylene.

The orientation of the magnets will depend on the application. When the north pole (−) (south seeking) side of the magnet is adjacent the body, the produced magnetic field assists in healing. When the south pole (+) (north seeking) side of the magnet is adjacent the body, the resultant magnetic field causes stimulation. There may be situations where the magnets may be placed in a mixed orientation. However at all times the appropriate spacing between magnets is maintained to achieve the maximum benefits.

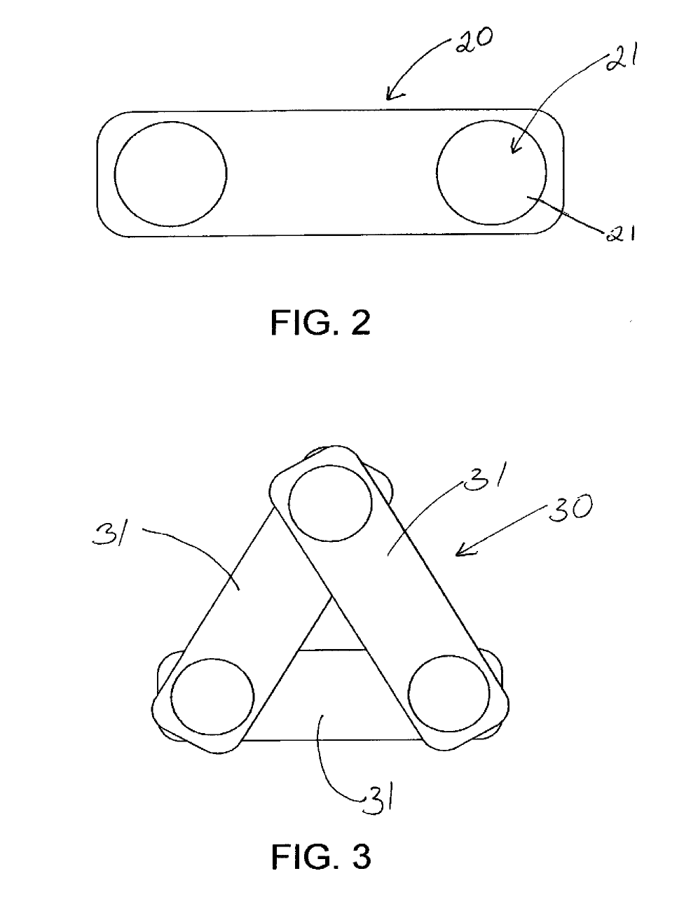

FIG. 3 shows a triangular arrangement 30 of the modular members 31. The modular members 31 are held in position by magnetic attraction. The triangular arrangement 30 assists in the relief of pain particularly period pain and is involved in pain management. Other geometrical shapes can be formed in a similar manner.

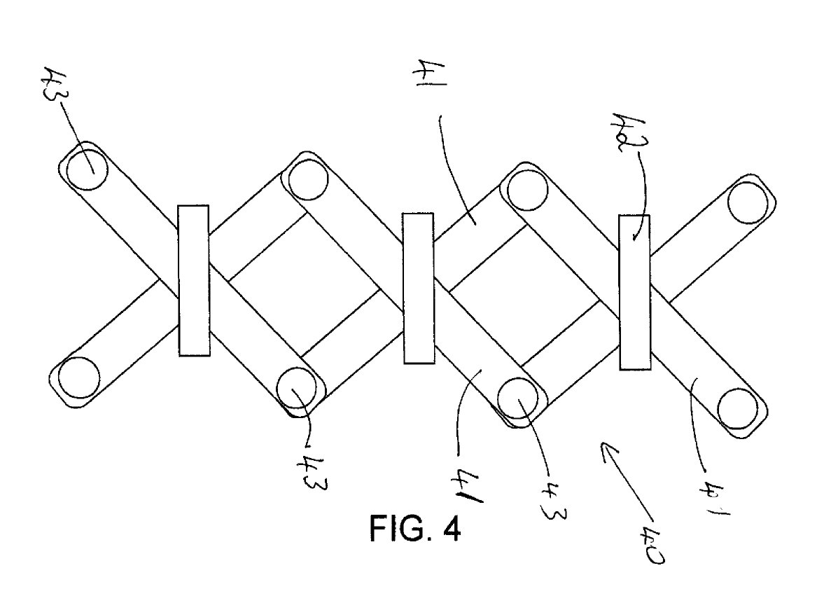

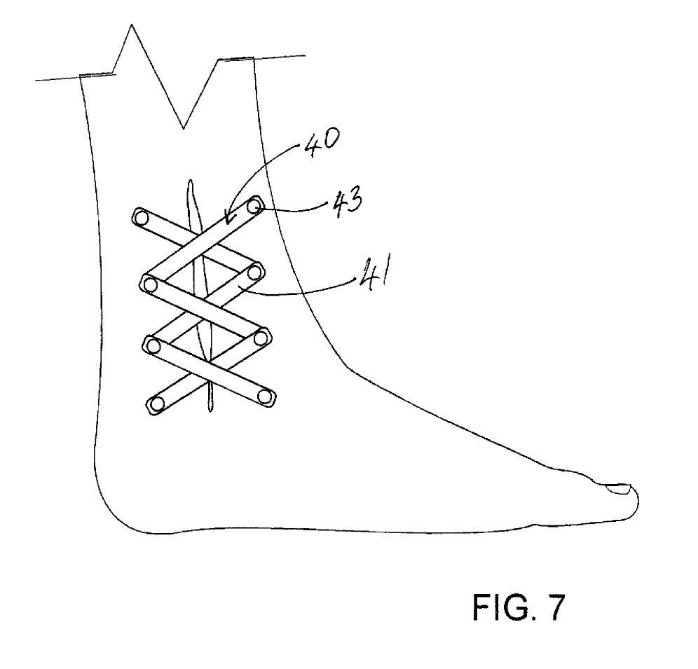

FIG. 4 shows a concertina arrangement 40 of the modular members 41. The modular members 41 are magnetically attached in a similar fashion to the triangular arrangement 30. The concertina arrangement 40 has a centrally-located slide 42 at the intersection of the modular members 41. The slides 42 have channels for the engagement of a connecting pin (not shown). Movement of the slide 42 up or down will extend or shorten the concertina arrangement 40. The concertina arrangement 40 or part there of can be made to form any desired shape with the use of the slides 42. In this way the concertina arrangement 40 can be shaped to follow the shape of a wound and assist in the healing. This is shown in FIG. 7. To assist in the healing of a wound, the magnets 43 in the concertina arrangement 40 are north pole orientated with the negative (north) side of the magnet adjacent the body portion.

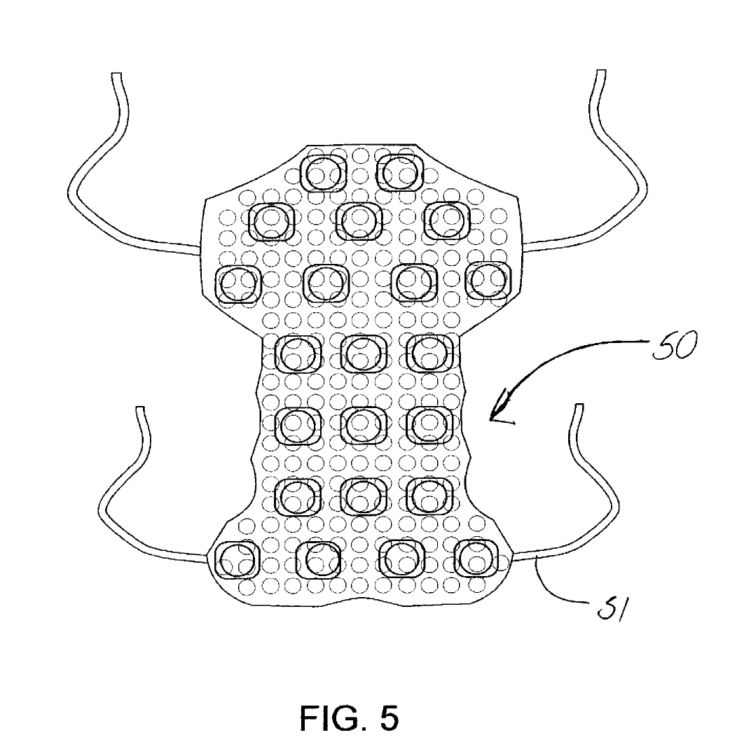

FIG. 5 is a magnetic mesh device 50 cut out in a desired pattern to assist in the relief of back pain. Straps 51 are provided to maintain the position of the mesh device 50 to achieve maximum therapeutic effects. The cut out pattern assists with the positioning of the device 50. The device 50 can be used as an overlay to clothes and under garments.

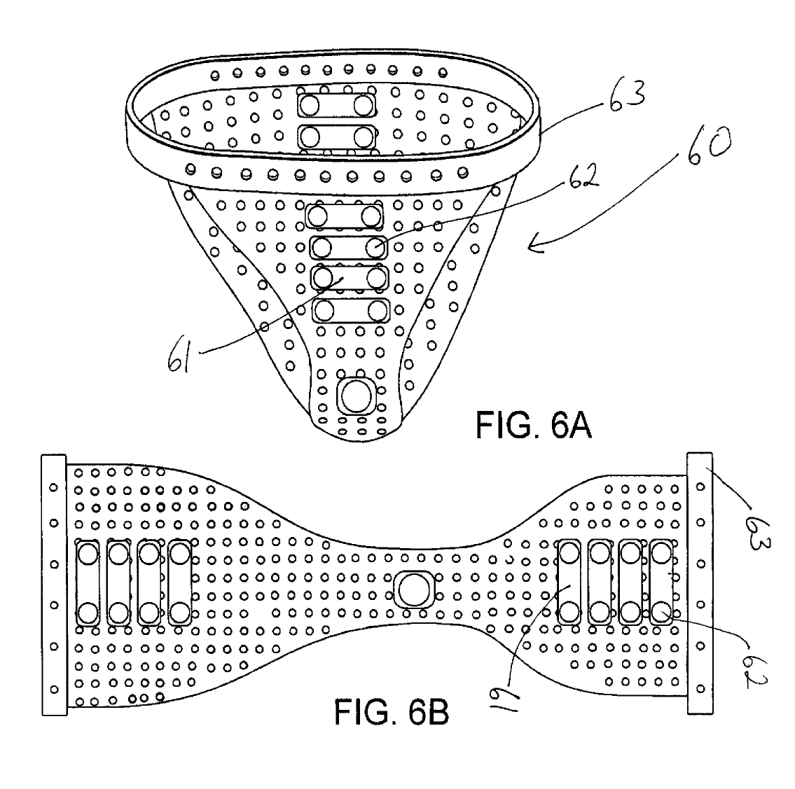

FIGS. 6A and 6B show a magnetic device 60 that can serve as an overlay to underpants and the like to provide relief to the condition of incontinence. The device 60 may also be used to provide relief from constipation, menstruation, urinary tract infections, lower back pain and vaginitis. The modular members 61 with their magnets 62 are position to be adjacent relevant acupuncture points when the device 60 is worn. The band 63 serves to hold the device 60 in position when worn. The band 63 also serves to align the device 60 with the relevant acupuncture points by locating the band above the belly button or above the top of the hip bone.

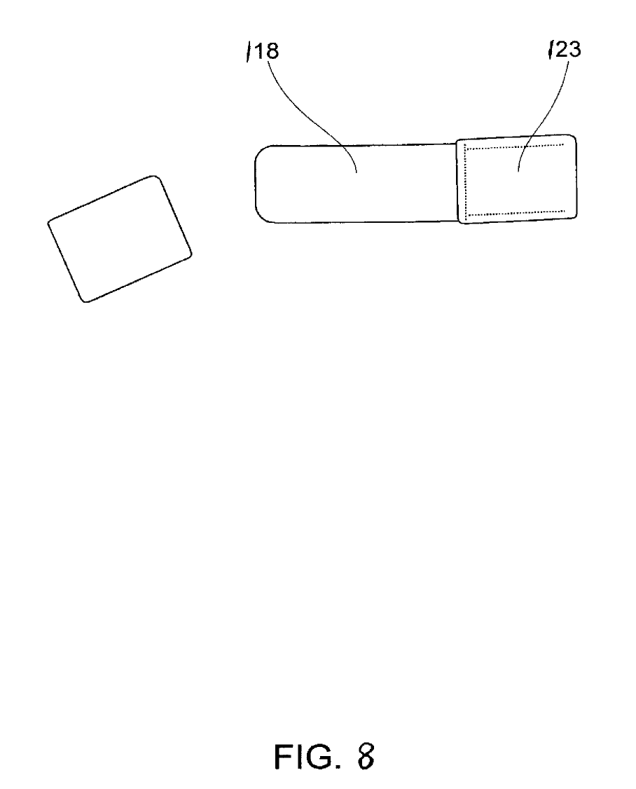

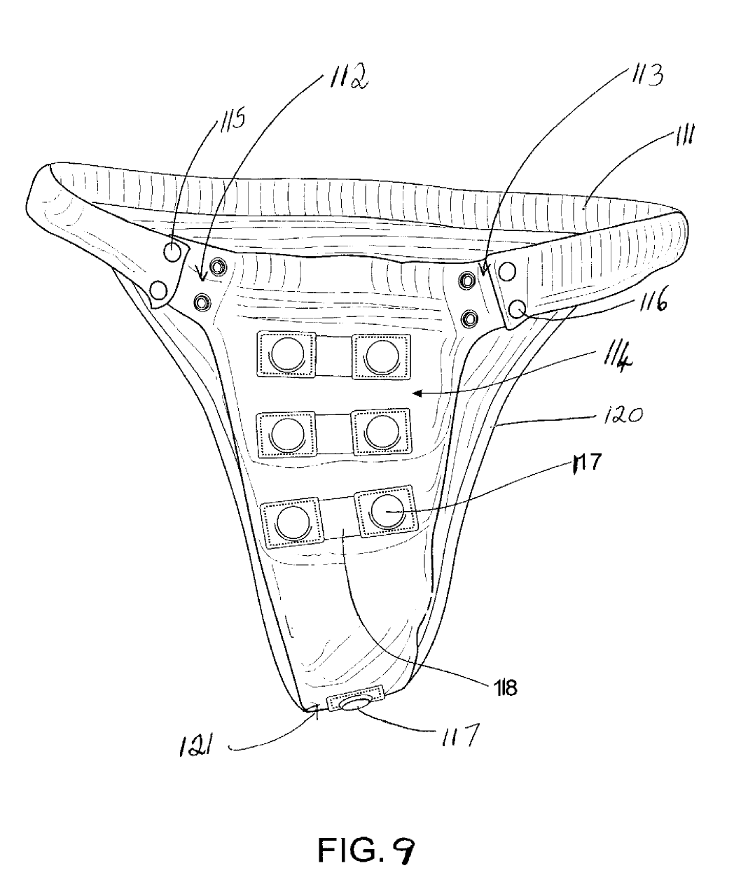

With reference to FIGS. 8 and 9, there is shown a garment overlay 110 for positioning over a pair of underpants. The garment overlay 110 is in the shape of a high leg-cut brief where the lower torso region is covered and the legs are unrestricted and not covered.

The garment overlay 110 has an elasticized band 111 for maintaining the position of the garment overlay 110 about the waist of the wearer. The band 111 also has two series of complementary press studs 112, 113 on either side of the front portion 114 of the garment overlay 110. Each series of complementary press studs 112, 113 consists of two spaced apart pairs of press studs 115, 116 for lengthening or shortening the length of the band 111. The ability to lengthen and shorten the band length is important so that a person can wear the garment overlay 110 over clothes of different thickness and the band 111 can be adjusted to accommodate different thickness from the use of different types or number of underclothes including absorbent pads. The ability to lengthen and shorten the band length is also important so that the band can be positioned above the hip bone in order to correctly align and orientate the magnets 117 particularly with respect to a pair of spaced apart magnets corresponding to L2 and L3 vertebrae in the spine.

The front portion 114 is substantially Y-shaped with three pairs of magnets 117 laterally spaced apart at a predetermined distance. The spacing between each pair of magnets 117 is predetermined to provide an optimal therapeutic treatment. The spacing between each pair of magnets 117 is maintained by a stiffener 118 which prevents the magnets from attaching to each other. The stiffener 118 is preferably made of a plastic and more preferably polypropylene.

The rear portion 120 is substantially Y-shaped with four pairs of laterally spaced apart magnets 117. Each pair of spaced apart magnets 117 is supported by a stiffener 118. The narrowest parts of the front portion 114 and rear portion 120 are joined to form the gusset portion 121. The gusset portion 121 has a single magnet 117 supported by a stiffener 118.

The cut of the front portion 114 and rear portion 120 in association with the band 111 provide a non restrictive garment that is relatively cool and comfortable to wear compared with other magnetic garments. Furthermore, the garment overlay 110 is relatively easy to put on because there are no restrictive leg portions.

The garment overlay 110 is manufactured by forming the garment without magnets. Having a plurality of stiffeners 118 which have a cloth cap 123 on each end and together are sewn in position on the garment. The one or more magnets 117 are then inserted into each cap 123 and the ends of each cap 123 are sewn to retain the magnets 117 in the desired position. The position of the magnets 117 is aligned with the relevant acupuncture points of a person.

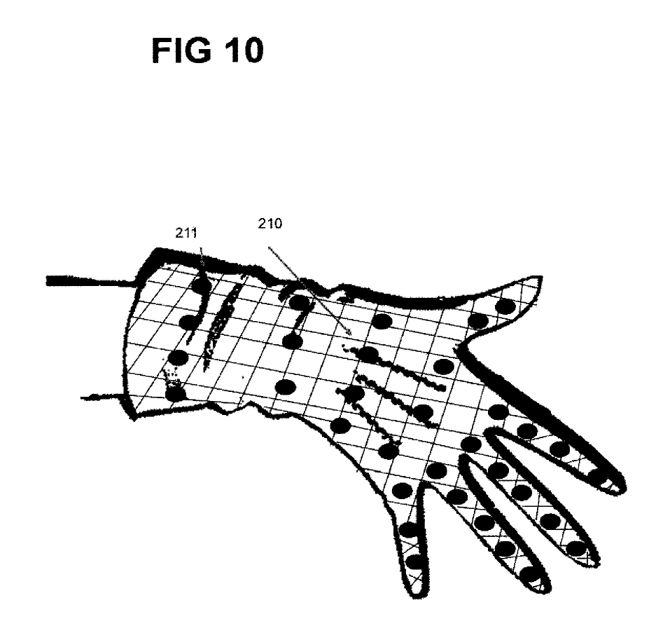

FIG. 10 shows a magnetic glove 210 comprising of a plastic woven mesh that is flexible to allow the hand and fingers to bend and grasp objects. The magnets 211 are embedded in the glove 210. The glove 210 is used to assist and or treat persons with carpal tunnel pain and problems and arthritis and rheumatic conditions.

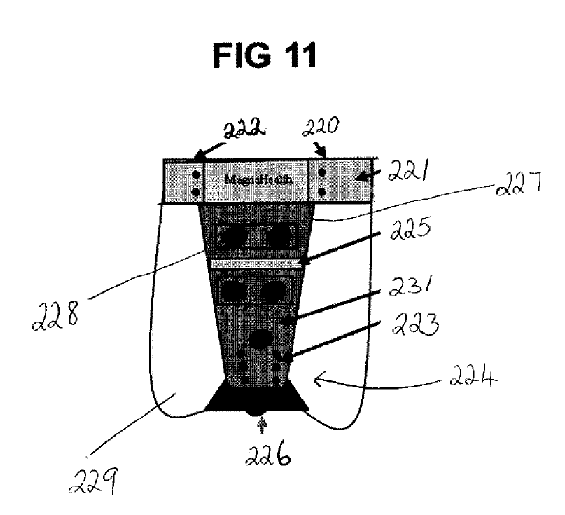

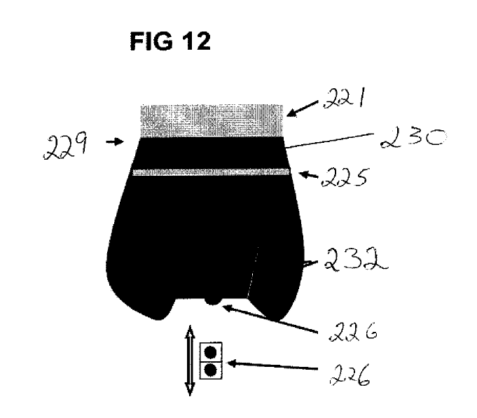

In FIGS. 11 and 12 there is shown an alternative incontinence overlay garment 220. The garment 220 has an adjustable wide waist band 221. The waist band 221 is preferably elasticized and has a press stud adjustment fitting 222. The garment 220 also has a series of fasteners 223 near the gusset 224 where the front material can be lengthened or shortened depending on the size and shape of the person in order to provide a comfortable fitting. The garment 220 may also be adjusted with an elasticized front and rear expander portions 225 which allows material to be vertically adjusted without causing the magnets to overlap. The perineum magnet 226 in the gusset 224 includes two 10 mm diameter and 5 mm thick spaced apart magnets. The pairs of magnets are 25 mm in diameter and 2.2 mm thick and have an N35 rating. The front section 227 has two pairs of magnets 228 while the rear section 229 has four pairs of magnets 230. The front section 227 also has a single magnet 231 of 25 mm diameter and 2.5 thickness and a N45 rating. The rear section 229 is also designed to cover the buttocks 232 to help maintain the position of the magnets and prevent them from coming together.

The garment 220 shown in FIGS. 11 and 12 may also be used to treat menstrual pain with the positioning of three sets of double strength magnets on the front section 227 and positioning of double strength magnets (such as two magnets) on front side sections at acupuncture points for menstrual pain.

In a similar way the garment 220 shown in FIGS. 11 and 12 may also be used to treat prostate inflammation with the positioning of three sets of double strength magnets on the front section 227. For treating sexual health, two double strength magnets may be used to replace the single magnet 231.

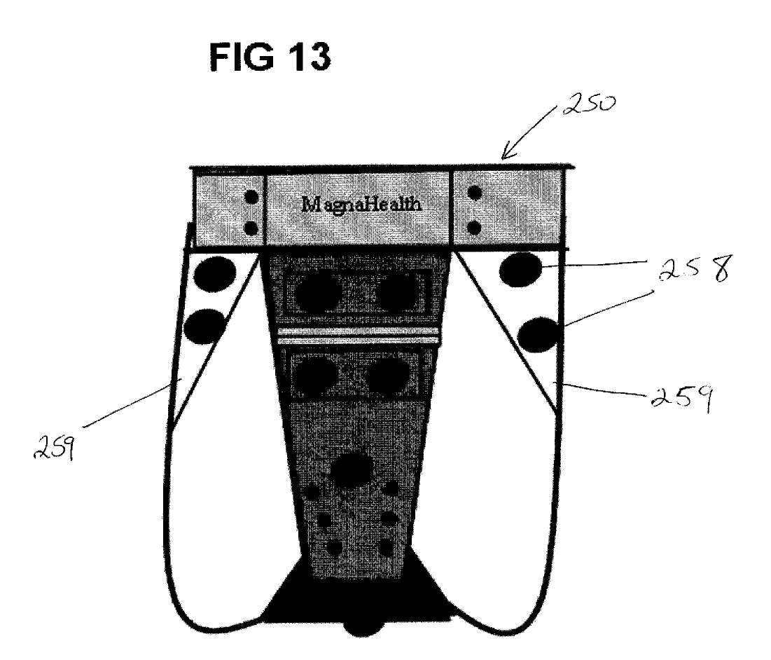

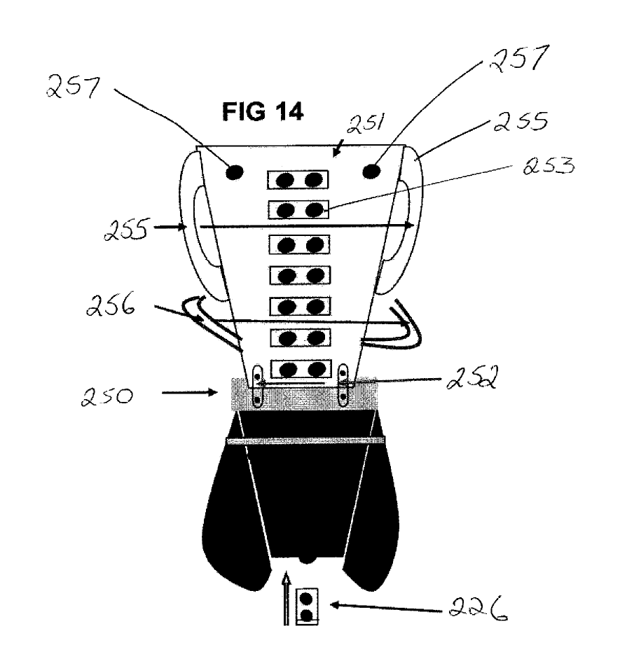

Upper and lower back pain can be treated with an underpants overlay garment 250 with an attachable back section 251 (as shown in FIGS. 13 and 14). The back section 251 is attached by means of straps 252. The back section 251 has seven sets of paired magnets 253 vertically positioned and aligned with the spine when worn. To maintain the position of the back section 251, it includes shoulder straps 255 and middle body straps 256. There are also two magnets 257 placed near the top and either side of the set of paired magnets 253. The underpants overlay garment 250 also has two magnets 258 on each front side portions 259.

Irritable bowl syndrome and pain in the hip and pelvic region can also be treated with the underpants overlay garment 250 and attachable back section 251 but without the two magnets 257 placed near the top and either side of the set of paired magnets 253.

### VARIATIONS

It will of course be realized that while the foregoing has been given by way of illustrative example of this invention, all such and other modifications and variations thereto as would be apparent to persons skilled in the art are deemed to fall within the broad scope and ambit of this invention as is herein set forth.

Throughout the description and claims this specification the word “comprise” and variations of that word such as “comprises” and “comprising”, are not intended to exclude other additives, components, integers or steps.

1-18. (canceled)

19. A therapeutic garment overlay wearable over underwear including
an adjustable member for adjusting the overlay about a body portion by lengthening or shortening the adjustable member thereby enabling the overlay to be fitted over the underwear irrespective of the thickness of the underwear;
locating means for locating the overlay about the body portion in an optimal therapeutic position;
cover member for supporting a plurality of magnets and shaped to be fitted over the underwear irrespective of the thickness of the underwear, each of said magnets is spaced from each other by a stiffener member that maintains the spacing of the magnets and substantially prevents joining of adjacent magnets because of magnetic attraction; wherein in use the garment overlay is able to position the magnets to provide a beneficial therapeutic effect irrespective of the underwear being worn.

20. A garment overlay as claimed in claim 19 wherein the adjustable member is a band that can be lengthened or shortened by means of adjustable fasteners; the locating means is the band shaped for locating the overlay over the wearer's hip bone; said cover member has a high cut leg so that the overlay can be fitted over the underwear.

21. A garment overlay as claimed in claim 19 wherein the cover member has a front portion, gusset and back portion; the adjustable member is an elasticized band that can be lengthened or shortened with two pairs of complementary press studs on either side of the front portion; the front and back portions are attached to the elasticized band to form an overlay that is in the shape of a high leg-cut brief where the lower torso region is covered and the legs are unrestricted and not covered.

22. A garment overlay as claimed in claim 19 wherein the cover member has a front portion, gusset and back portion; the front portion is substantially Y-shaped with three pairs of magnets laterally spaced apart at a predetermined distance; the rear portion is substantially Y-shaped with four pairs of magnets laterally spaced apart at a predetermined distance; the gusset portion has a magnet; the adjustable member is an elasticized band that can be lengthened or shortened with two pairs of complementary press studs on either side of the front portion; the front and back portions are attached to the elasticized band to form an overlay that is in the shape of a high leg-cut brief where the lower torso region is covered and the legs are unrestricted and not covered, said formed overlay is used for therapeutically treatment of incontinence.

23. A garment overlay as claimed in claim 19 wherein the overlay is formed with stiffener members that have a cloth cap at each end for the insertion of one or more magnets in each cloth cap.

24. A garment overlay as claimed in claim 19 wherein the stiffener members are fixed in a position that aligns the magnets with acupuncture points on the body.

25. A garment overlay as claimed in claim 19 wherein there are elasticized front and rear portions in the cover member to vertically adjust the cover member without causing the magnets to overlap.

26. A garment overlay as claimed in claim 19 wherein a back section with a plurality of stiffener members with magnets is attachable to the overlay and positioned to cover the spine to provide a therapeutic effect.

27. A garment overlay as claimed in claim 19 wherein the cover member has a front portion, gusset and back portion; the front portion is substantially Y-shaped with three pairs of magnets laterally spaced apart at a predetermined distance and two magnets on front side sections at acupuncture points for the treatment of menstrual pain.

28. A garment overlay as claimed in claim 19 wherein the cover member has a front portion, gusset and back portion; the front portion is substantially Y-shaped with three pairs of magnets laterally spaced apart at a predetermined distance for the treatment of prostrate inflammation.

29. A garment overlay for positioning over underwear worn by a wearer, including
an adjustable band that has a plurality of fasteners for lengthening or shortening the band;
a cover member shaped to cover the lower torso region without covering the legs, a plurality of magnets fixed to the cover member and supported in position by a stiffener member, wherein said band is shaped to fit around a wearer's waist and over the wearer's hip bones for location purposes to align and orientate said magnets to provide a beneficial therapeutic effect.

30. A garment overlay for positioning over underwear worn by a wearer as claimed in claim 29 wherein there are three pairs of laterally spaced apart magnets on a front section of the overlay portion, four pairs of laterally spaced apart magnets on a rear section of the overlay portion and there is a single magnet fixed at the gusset section of the overlay portion; said overlay portion is in the shape of a high leg-cut type garment with ample material to overlay the underwear.
